(19) United States
(12) Patent Application Publication
(10) Pub. No.: US 2009/0326653 A1
(43) Pub. Date: Dec. 31, 2009

(54) BIST (Breast Implant Softening Technology)

(76) Inventor: S. L. Tribby, Grants Pass, OR (US)

Correspondence Address: S. L. TRIBBY, 233 ROGUE RIVER HWY #199, GRANTS PASS, OR, 97527, US

(21) Appl. No.: 12/455,289

(22) Filed: Jul. 17, 2009

(51) Int. Cl. A61F 2/12; B29C 71/00

(52) U.S. Cl. 623/8; 264/340

(57) ABSTRACT

The Method of USING holograms:

Any and all holograms

No matter what material they are made of

No matter what machine they are made with

No matter their type, size, shape, design, image, color, texture, material, thickness, dimension or style

No matter where they originated from

No matter what technology is being used on them

No matter their original purpose being used for

Whether for one or multiple purposes being used for

If alone or with other products

For the purpose of USING holograms to soften, maintain and or restore softness, flexibility and pliability of any and all breast implants whether being used on the breast or being used on the breast implant.

BIST applies to USING hologram(s) for softening, maintaining and or restoring softness, flexibility and pliability of breast implants including but not limited to breast implants made of silicone-gel, saline-filled or any and all other materials or substances, listed or not listed, known or unknown.

The method of USING hologram(s), any and all hologram(s) for softening breast implants, including but not limited to holograms which are stickers, stamps, patches, protective pockets, pouches, packets, artwork design or display, animated, metalized, 2D/3D, flip flop, film, lenticular holograms (including but not limited to lenticular flip, 3D, morph, zoom, motion or multi-effect), simple hand embossed, foil, stainless steel, gold, silver, sterling silver, electroplate, seals, badges, masters, security cards, tape or strips, IDs, ID overlays, sequin, mobile decals, hot stamps, stamps, general holograms, labels, labeling, customized, sew-on, press-on, iron-on, clip-on, stick on, transparent, tape, paper, fabric, scratch or scratch off, stationary or floating images, kinetic movement holograms, pearlescent effect, revolving effect, projective ray effect, electromagnetic, laser beam hidden text or image, mole hidden text or image, micro text, wearable holograms, laid on, put in, wrapped around, attached to or embedded in or on any product, object or just by itself, holograms with or without visual depth, original holograms, copies, masters, film, templates, glass or duplicate holograms, made of anything else, listed or not listed, known or unknown;

It doesn't matter what original purpose or how the hologram(s) are being used for, including but not limited to researching of softening breast implants, clinical trials studying, testing, theory applying, laboratory experimenting, actually using them in, by, around, over, under, near, or on the breast or on the breast implant, listed or not listed, known or unknown;

Holograms being made of any and all materials including but not limited to paper, plastic, mylar, stainless steel, sterling silver, silver, gold, metal, shim, sheath, fabric, organic or non-organic, leather, foil, fur, metallic, cellophane, mineral, and or made of anything else, listed or not listed, known or unknown;

Holograms being made with or by any and all machines, software or manually by hand, including but not limited to dot-matrix machines, 2D/3D machines, laminator, glass masters, recombination machines, laser light, laser beam, pulsed laser, animated, narrow hard web embosser, wide web soft embosser, computer software controlled, kinetic movement machines, projective ray, hot melt adhesive coating machines, electronic forming, automatic die-cutting, electromagnetic, or with, by any and all other machines and or any other methods of making holograms, listed or not listed, known or unknown, no matter where the machines originated or are located, where the holograms originated from, what country the machines are located in, what machines the holograms came from, who or what is operating the machines that make the holograms, or who or how the formula technology is entered into the machines or onto the holograms, listed or not listed, known or unknown;

Whether the hologram(s) are being placed, attached, pinned, stapled, snapped, hooked, pierced, tattooed, glued, taped, velcroed to, with, or directly on the breast, near, around, above, over, or under the breast with any tape, adhesive, stick-on, press-on, glue stick, static cling, or any other method of placing, putting, adhering or attaching hologram(s) with glue, paste, velcro, magnetically, electronically or electromagnetically to, on, above, around or near the breast, listed or unlisted, known or unknown;

Whether the hologram(s) are being placed, attached, or adhered directly on the breast implant, being embedded in the breast implant, being placed inside the breast implant, being placed inside, on or with another object, product, item, and then being placed on, being embedded in, or being placed inside the breast implant, listed or not listed, known or unknown;

Whether the hologram(s) are being placed, attached, adhered, embedded in, on, or to breast implant(s) which are already implanted within the breast, chest, body, or outside the body not yet implanted, awaiting to be implanted, or being used for breast implant(s) which are in a laboratory, clinical trial, testing, research, manufacturing facility, warehouse, storage, packaged for shipping, or any and all other stages of processing, manufacturing, and or completing breast implants, listed or not listed, known or unknown;

Whether the hologram(s) are being attached, pinned, stapled, snapped, hooked, glued, taped, velcroed to, with, or on a bra, sports bra, padding, pasties, tassel, stick-on or press-on bra or breast support, breast lifter, push-up, under-wire, tag, label, article of clothing, object, item, or product, listed or not listed, known or unknown; or whether the hologram(s) are being attached, pinned, stapled, snapped, hooked, glued, taped, velcroed to, with or on another object, product, jewelry, or item of any kind and being placed in, over, under, with or on a bra, sports bra, padding, pasties, tassel, stick-on or press-on bra or breast support, breast lifter, push-up, under-wire, tag, label, article of clothing, object, item, or product, listed or not listed, known or unknown;

No matter if the hologram(s) are being sewn into or onto a bra, sports bra, padding, pasties, tassel, stick-on or press-on bra or breast support, breast lifter, push-up, under-wire, tag, label, article of clothing, object, item, or product, listed or not listed, known or unknown;

Hologram(s) that are being sewn inside a blouse, shirt, vest, jacket, scarf, choker, necklace, jewelry, armband, chest wrap, cover, or any other garment, product, object, item, listed or not listed, known or known, which is placing the hologram(s) over, under, near, against, around, above, or on the breast, neck, underarms, arms, back, chest, ribs, or upper body above the waist, listed or unlisted, known or unknown;

Hologram(s) may be hanging on, from or with any bra, sports bra, padding, pasties, tassel, stick-on or press-on bra or breast support, breast lifter, push-up, under-wire, tag, label, article of clothing, object, item, or product, listed or not listed, known or unknown;

Whether the hologram(s) are being placed inside or on a protective sheath, sleeve, pocket or packet of cardboard, paper, metal, cellophane, or any other material, product, object, item and being worn inside, on, with or hanging from any bra, sports bra, padding, pasties, tassel, stick-on or press-on bra or breast support, breast lifter, push-up, under-wire, tag, label, article of clothing, object, item, or product, listed or not listed, known or unknown.

1. I hereby CLAIM that BIST (Breast Implant Softening Technology) applies to the method of USING hologram(s), any and all holograms and/or USING hologram technology by placing them on the breast(s) with breast implant(s), placing then on another product(s) then placing them on the breast(s) with breast implant(s), placing them directly on the breast implant(s), placing them on another product(s) then placing them directly on the breast implant(s), or embedding them directly into the breast implant(s) or embedding them into another product(s) then placing them on the breast(s); the hologram(s) and/or hologram technology can and will soften, maintain and/or restore the softness, flexibility and pliability of any and all breast implant(s) whether the hologram(s) and/or hologram technology is being used on the breast(s) or being used on or embedded inside the breast implant(s) whether the breast implant(s) are inside the body or outside of the body.
